(19) United States
(12) Patent Application Publication
(10) Pub. No.: US 2025/0332436 A1
(43) Pub. Date: Oct. 30, 2025

(54) PHOTOTHERAPY PAD

(71) Applicant: Shenzhen Kaiyan Medical Equipment Co., Ltd., Shenzhen (CN)

(72) Inventors: Alain Dijkstra, Amstelveen (NL); LI XIANG, Shenzhen (CN)

(73) Assignee: Shenzhen Kaiyan Medical Equipment Co., Ltd., Shenzhen (CN)

(21) Appl. No.: 19/040,888

(22) Filed: Jan. 30, 2025

(30) Foreign Application Priority Data
Apr. 25, 2024 (CN) 2024208916640

(51) Int. Cl. A61N 5/06

(52) U.S. Cl. CPC A61N 5/0613; A61N 2005/0645; A61N 2005/0651; A61N 2005/0659; A61N 2005/0663

(57) ABSTRACT

Embodiments of the present invention disclose a flexible phototherapy pad designed to enhance comfort and adaptability for treatment areas. The pad includes a main body area adapted to a treatment area, along with a first edge area and a second edge area, each of which can be folded along the respective sides of the main body area. Both the first edge area and the second edge area are connected to the main body and extend along the adjacent sides. The flexibility of the first edge area and the second edge area areas allows the pad to conform to the complex curved surface of the human body, addressing the fit and comfort issues commonly encountered with conventional phototherapy pads. The design of the phototherapy pad provides significant improvements in practicality and user comfort, offering high value in therapeutic applications.

### TECHNICAL FIELD

The present invention relates generally to the technical field of phototherapy equipment, specifically to a phototherapy pad that is adaptable to fit to groin and perineum area of the human body.

### BACKGROUND ART

With the continuous advancement of medical technology, therapeutic physical therapy equipment has become increasingly prevalent in clinical practice. Among the various modalities, phototherapy has gained significant attention for its non-invasive, painless, and radiation-free benefits. It is commonly used in wound healing, sterilization, and disinfection, as well as for pain and itching relief.

However, most existing phototherapy devices are designed as flexible pads with regular shapes, intended for localized light therapy. While some designs have made efforts to better fit specific body parts, they still have notable shortcomings in practical applications. Although these devices achieve some degree of body part wrapping, their overall flexibility and adaptability remain limited. This issue is particularly evident when the device is applied to complex, curved areas of the body, where the fit and effectiveness of the phototherapy pads often fall short.

A common scenario in clinical practice is the use of phototherapy devices following surgical procedures, particularly in areas like the groin and perineum. For example, after perineal surgery, the affected areas are shaved and require light-based sterilization, disinfection, and pain relief for several days. Existing flexible pads, with their regular shapes, are not well-suited to conform to the intricate curves of the groin and perineum. This lack of proper fit not only reduces treatment efficacy but also leads to discomfort, particularly at the edges of the pads, which may squeeze the skin during prolonged use. Moreover, when a user walks, the flexible pads at their edges rubs against the legs of the user causing discomfort while use.

There is a clear need in the field for a more adaptable and comfortable phototherapy device. Such a device should be designed to accommodate the unique shapes of different body parts, especially in challenging areas like the groin and perineum. To address these issues, the device should offer higher flexibility and plasticity, allowing it to conform to the body's local morphology for a more personalized and comfortable treatment experience. The edges of the device are designed to provide comfort to the user even when the user is in motion. Furthermore, targeted structural design is needed to mitigate the pressure exerted by the device's edges, preventing discomfort and improving overall usability.

### OBJECTS OF THE INVENTION

Some of the objects of the invention are as follows:

An object of the present invention is to provide a phototherapy pad that is adaptable and can fit the complex curved surface of the human body.

Another object of the present invention is to provide a phototherapy pad having a bendable edge structure to adapt to the curvature of complex body parts such as the groin and perineum.

Another object of the present invention is to provide a phototherapy pad that adopts a multi-layer flexible structure of a light transmitting layer and the overall fit and wearing comfort of the phototherapy pad are updated and upgraded from the material to the process.

Another object of the present invention is to provide a phototherapy pad with a flexible soft convex ring that protects the fragile light-emitting unit when the phototherapy pad is bent and deformed.

Another object of the present invention is to provide a phototherapy pad in which the flexible soft convex ring reduces the blind spot of light with the help of an optical structure while improving the uniformity of irradiation and ensuring the therapeutic effect.

Another object of the present invention is to provide a phototherapy pad with an in-built control module and fixing device that provide precise parameter control while firmly fixing the phototherapy pad to the body part.

### SUMMARY OF THE INVENTION

According to a first aspect of the present invention, a phototherapy pad that is adaptable and can fit the complex curved surface of the human body is provided. The phototherapy pad comprises: a main body area configured to cover a treatment area, the main body area comprises an upper end, a lower end a first side, and a second side connecting the upper end and the lower end; a first edge area having a first end connected to the upper end of the main body area and extending laterally along the first side of the main body area towards the lower end; and a second edge area having a first end connected to the upper end of the main body area and extending laterally along the second side of the main body area towards the lower end.

In one embodiment of the invention, the main body area further comprises a first transition zone located on the first side of the main body area, the first edge area is connected to the first transition zone; and width of the first transition zone is less than or equal to the width of the first edge area.

In one embodiment of the invention, the main body area further comprises a second transition zone located on the second side of the main body area, the second edge area is connected to the second transition zone; and width of the second transition zone is less than or equal to the width of the second edge area.

In one embodiment of the invention, the main body further comprises a first connecting portion at the first side and a second connecting portion at the second side on the upper end of the main body area.

In one embodiment of the invention, the phototherapy pad further comprising a fixing belt to secure the phototherapy pad, the fixing belt is connected to the first connecting portion and the second connecting portion.

In one embodiment of the invention, the phototherapy pad comprises a light-transmitting layer, a substrate layer, and a light source assembly for providing phototherapy; the substrate layer is connected to the light-transmitting layer, and the light source assembly is installed between the light-transmitting layer and the substrate layer.

In one embodiment of the invention, the light-transmitting layer and the substrate layer are both flexible; and the light source assembly comprises a flexible LED light board or LED light strip.

In one embodiment of the invention, the light-transmitting layer comprises a convex ring at side facing the substrate layer, and a light-emitting unit is positioned at the center of the convex ring.

In one embodiment of the invention, a positioning hole is provided in the light source assembly, and a positioning protrusion is provided on the substrate layer to fit into the positioning hole.

In one embodiment of the invention, a control assembly is provided in the first edge area or the second edge area, the control assembly is electrically connected to the light source assembly.

In the context of the specification, the term “processor” refers to one or more of a microprocessor, a microcontroller, a general-purpose processor, a Field Programmable Gate Array (FPGA), or an Application Specific Integrated Circuit (ASIC), and the like.

In the context of the specification, the phrase “memory unit” refers to volatile storage memory, such as Static Random Access Memory (SRAM) and Dynamic Random Access Memory (DRAM) of types such as Asynchronous DRAM, Synchronous DRAM, Double Data Rate SDRAM, Rambus DRAM, and Cache DRAM, etc.

In the context of the specification, the phrase “storage device” refers to a non-volatile storage memory such as EPROM, EEPROM, flash memory, or the like.

In the context of the specification, the phrase “communication interface” refers to a device or a module enabling direct connectivity via wires and connectors such as USB, HDMI, VGA, or wireless connectivity such as Bluetooth or Wi-Fi, or Local Area Network (LAN) or Wide Area Network (WAN) implemented through TCP/IP, IEEE 802.x, GSM, CDMA, LTE, or other equivalent protocols.

In the context of this specification, terms like “light”, “radiation”, “irradiation”, “emission” and “illumination”, etc. refer to electromagnetic radiation in frequency ranges varying from the visible frequencies to Infrared (IR) frequencies and wavelengths, wherein the range is inclusive of visible light, and IR frequencies and wavelengths. Preferably, it refers to low-level electromagnetic radiation of low-level red and near-infrared (NIR) light. It is to be noted here that IR radiation can be categorized into several categories according to respective wavelength ranges which are again envisaged to be within the scope of this invention. A commonly used subdivision scheme for IR radiation includes Near IR (0.75-1.4 μm), Short-Wavelength IR (1.4-3 μm), Mid-Wavelength IR (3-8 μm), Long-Wavelength IR (8-15 μm) and Far IR (15-1000 μm). In this regard, light application is at relatively low energy densities, typically below about 500 mW, as compared to other forms of laser therapy that are used for ablation, cutting, and thermally coagulating tissue. In some instances, electromagnetic radiation can also be in wavelengths in the blue or ultraviolet regions, especially for treatment of conditions that occur at the skin surface, such as psoriasis or infection.

In the context of the specification, when an element is referred to as being “fixed to” or “disposed to” another element, it may either be directly on another element or indirectly on that other element. When a component is said to be “connected” or “connected to” another component, it may be directly connected to another component or indirectly connected to other components on the piece.

In the context of the specification, the terms “first’, “second” and “third” are only used for descriptive purposes and do not implicate the relative importance or implicitly indicate the quantity of technical features indicated.

In the context of the specification, the term “plurality” means two or more than two, unless otherwise indicated.

In the context of the specification, the term “several” means more than one, unless otherwise specified.

In the context of the specification, “Light Emitting Diodes (LEDs)” refer to semiconductor diodes capable of emitting electromagnetic radiation when supplied with an electric current. The LEDs are characterized by their superior power efficiencies, smaller sizes, rapidity in switching, physical robustness, and longevity when compared with incandescent or fluorescent lamps. In that regard, the one or more LEDs may be through-hole type LEDs (generally used to produce electromagnetic radiations of red, green, yellow, blue, and white colors), Surface Mount Technology (SMT) LEDs, Bi-color LEDs, Pulse Width Modulated RGB (Red-Green-Blue) LEDs, and high-power LEDs, etc.

Materials used in one or more LEDs may vary from one embodiment to another depending upon the frequency of radiation required. Different frequencies can be obtained from LEDs made from pure or doped semiconductor materials. Commonly used semiconductor materials include nitrides of Silicon, Gallium, Aluminum, Boron, Zinc Selenide, etc. in pure form or doped with elements such as Aluminum and Indium, etc. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used to generate a wider color gamut. White and other colored lightings may also be produced using phosphor coatings such as Yttrium Aluminum Garnet (YAG) in combination with a blue LED to generate white light and Magnesium doped potassium fluorosilicate in combination with blue LED to generate red light. Additionally, near Ultraviolet (UV) LEDs may be combined with europium-based phosphors to generate red and blue lights and copper and zinc doped zinc sulfide-based phosphors to generate green light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, a discussion on the generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety, by reference.

In several embodiments, one or more LEDs may also be micro-LEDs described through U.S. Pat. Nos. 8,809,126 B2, 8,846,457 B2, 8,852,467 B2, 8,415,879 B2, 8,877,101 B2, 9,018,833 B2 and their respective family members, assigned to Nth Degree Technologies Worldwide Inc., which are included herein by reference, in their entirety. The one or more LEDs, in that regard, may be provided as a printable composition of the micro-LEDs, printed on a substrate.

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings wherein like reference letters and numerals indicate the corresponding parts in various figures in the accompanying drawings, and in which:

FIG. 1 shows a three-dimensional structure of a phototherapy pad, in accordance with an embodiment of the present invention.

FIG. 2 shows an exploded view of the phototherapy pad, in accordance with an embodiment of the present invention.

FIG. 3 shows a light-transmitting layer of the phototherapy pad, in accordance with an embodiment of the present invention.

FIG. 4 shows a main body area of the phototherapy pad without a fixing belt, in accordance with an embodiment of the present invention.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

The terms “a” and “an” herein do not denote a limitation of quantity, but rather denote the presence of at least one of the referenced items. The terms “having”, “comprising”, “including”, and variations thereof signifies the presence of a component.

In the description of the embodiments of the present application, it is to be understood that the orientations or positional relationships indicated by the terms “length”, “width”, “upper”, “down”, “front”, “rear”, “left”, “right”, “vertical”, “horizontal”, “top”, “bottom”, “inner”, “outer”, etc., are based on the orientations or positional relationships shown in the drawings and are only for the convenience of describing the embodiments of the present application and for simplifying the description, and are not intended to indicate or imply that the device or element referred to must have a particular orientation, be constructed and operated in a particular orientation, Therefore, it cannot be construed as a limitation on the present application.

In addition, the terms “first” and “second” are used for descriptive purposes only and should not be construed as indicating or implying relative importance or implying the number of technical features indicated. Thus, defining the “first” and “second” features may explicitly or implicitly include one or more of these features. In the description of the embodiments of the present application, “plurality” means two or more of them, unless otherwise expressly and specifically qualified.

In the embodiments of the present application, unless otherwise expressly specified and limited, the terms “mounted”, “connected”, “connected”, “fixed”, etc., shall be construed broadly, for example, they may be fixed, detachable, or integral; It can be mechanically or electrically connected; It can be directly connected or indirectly connected through an intermediate medium, it can be the internal connection of two elements or the interaction relationship between two elements. For those of ordinary skill in the art, the specific meaning of the above terms in the embodiments of the present application may be understood according to the specific circumstances.

In the embodiments of the present application, unless otherwise expressly specified and limited, the first feature “above” or “below” the second feature can be that the first and second features are in direct contact, or the first and second features are in indirect contact through an intermediate medium. Moreover, the first feature “above”, the second feature can mean that the first feature is directly above or obliquely above the second feature, or simply means that the first feature is higher in level than the second feature. The first feature “below” the second feature can mean that the first feature is directly below or obliquely below the second feature, or simply means that the first feature is lower in level than the second feature.

Embodiments of the present invention disclose a phototherapy pad with a structure that enables it to flexibly adapt to the curvature of various parts of the human body through a bendable edge structure, especially for complex areas such as the groin and perineum. It has a significant fit advantage. The phototherapy pad adopts a multi-layer flexible structure of a light-transmitting layer, a light source component, and a substrate layer stacked from the inside to the outside, and the overall fit and wearing comfort of the phototherapy pad are updated and upgraded from the material to the process. In addition, a flexible soft convex ring is designed between the light-transmitting layer and the light source component, which not only provides protection to the fragile light-emitting unit when the phototherapy pad is bent and deformed but also can reduce the blind spot of light with the help of a special optical structure while improving the uniformity of irradiation and ensuring the therapeutic effect. The phototherapy pad has a built-in control module and fixing device that can provide precise parameter control while firmly fixing the phototherapy device to the part that needs treatment.

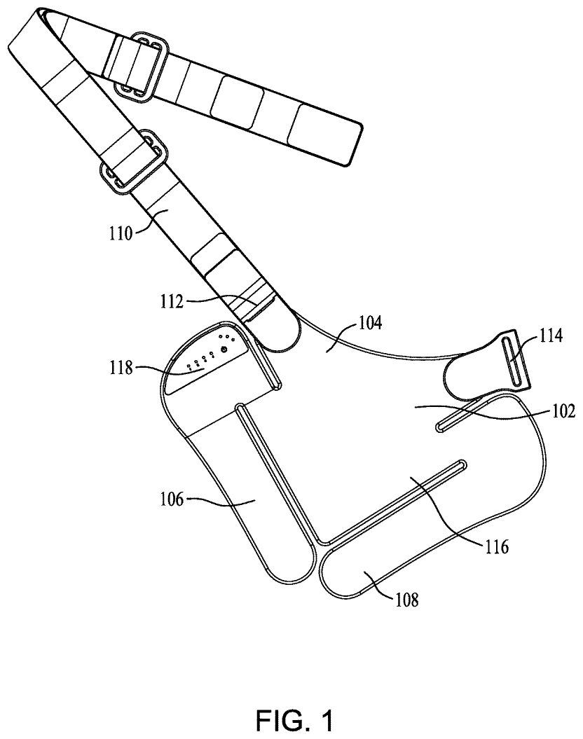

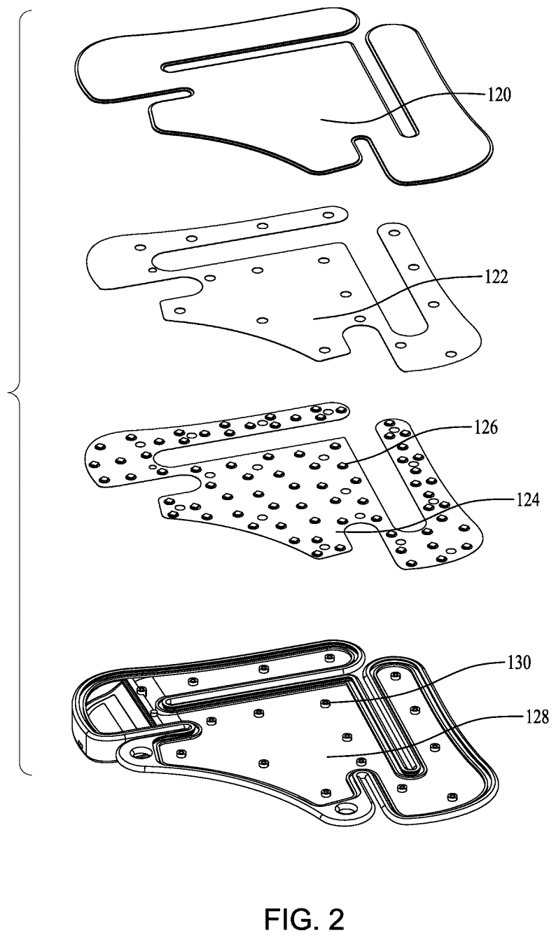

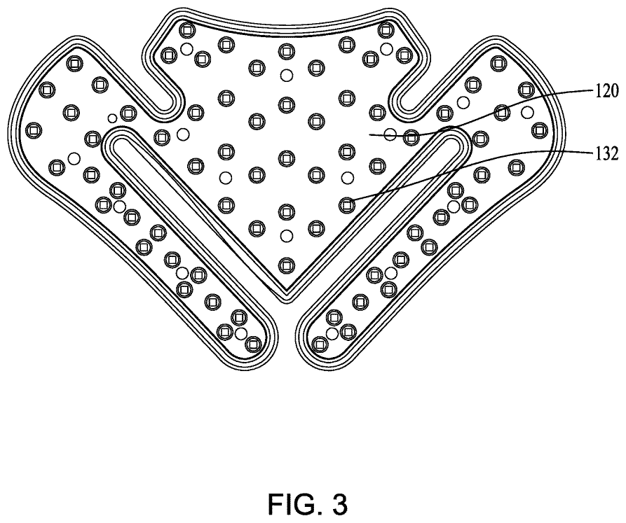

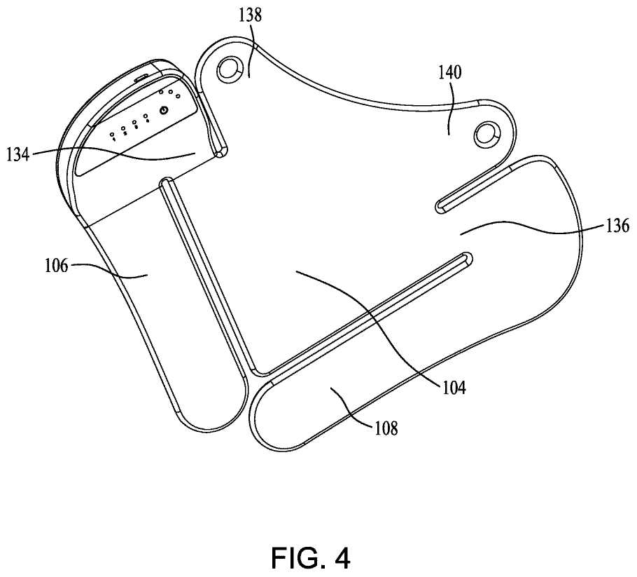

Several embodiments of the present invention will now be described in detail with references to FIGS. 1 to 4.

Referring to FIGS. 1 to 4, a phototherapy pad 102 is provided. The phototherapy pad 102 includes a main body area 104 designed to conform to the treatment area, a first edge area 106 that can be folded along a first side of the main body area 104, and a second edge area 108 that can be folded along a second side of the main body area 104. One end of the first edge area 106 is attached to the main body area 104 and extends along the first side. Similarly, one end of the second edge area 108 is attached to the main body area 104 and extends along the second side. The first edge area 106 and the second edge area 108 are symmetrical in shape and can be bent and deformed relative to the main body area 104.

The shape and size of the main body area 104 can be designed according to specific treatment needs to achieve the best adaptation to different human treatment areas. The main body area 104 comprises an upper end and a lower end. One end of the first edge area 106 and the second edge area 108 are respectively connected to the main body area 104 at the edges on the first side and the second side of the upper end and are extended along the first side and the second side of the main body area 104. The other end of the first edge area 106 and the second edge area 108 are freely floating such that there is a gap between the first side of the main body area 104 and the first edge area 106. Similarly, there is a gap between the second side and the second edge area 108. The main function of the first edge area 106 and the second edge area 108 is to be folded along the end connected to the main body area 104, so that the phototherapy pad 102 can better fit the curved surface of the body and improve the wearing comfort.

Referring to FIGS. 1 to 4, the width of the phototherapy pad 102 gradually decreases from the end of the main body area 104 connected to the first edge area 106 (or the second edge area 108) to the other end of the main body area 104. The width of the phototherapy pad 102 gradually decreases from the upper end of the main body area 104 to the lower end of the main body area 104. Optionally, the main body area 104 can be roughly inverted triangle, and the first edge area 106 and the second edge area 108 can be respectively extended downward along the two sides of the main body area 104, such that the phototherapy pad 102 is roughly triangular. Only one end of the first edge area 106 and the second edge area 108 is connected to the main body area 104. The first edge area 106 and the second edge area 108 extends along the length of the first side and second side of the main body area 104 such that there is a gap between the first edge area 106 and the main body area 104. Similarly, there is a gap between the second edge area 108 and the main body area 104 along the length of the second side of the main body area 104. In this way, it can be better adapted to the wearing area of the human body.

A first transition zone 134 is provided on the first side of upper edge of the main body area 104. The first transition zone 134 extends in a direction away from the free end of the first edge area 106. The end of the first transition zone 134 away from the main body area 104 is connected to the first edge area 106. The height of the first transition zone 134 is less than or equal to the length of the first edge area 106. Optionally, the first transition zone 134 is arranged as a long rectangular strip as a whole, and its length direction is parallel to the bottom edge of the main body area 104. The end of the first transition zone 134 away from the main body area 104 is connected to the first edge area 106. The height of the first transition zone 134 is less than or equal to the length of the first edge area 106 and extends in a direction away from the free end of the first edge area 106. The free end of the first transition zone 134 is in an arc shape. The length of the first transition zone 134 is greater than the width of the first edge area 106. The height of the first transition zone 134 corresponds to the height from the end connected to the first edge area 106 to the free end which is arc shaped. The length of the first transition zone 134 corresponds to the length from the end connected to the main body area 104 to the end connected to the first edge area 106.

A second transition zone 136 is provided on the other side of the main body area 104. The second transition zone 136 extends in a direction away from the free end of the first edge area 106. The end of the second transition zone 136 away from the main body area 104 is connected to the second edge area 108. The height of the second transition zone 136 is less than or equal to the length of the second edge area 108. Optionally, the second transition zone 136 is arranged in a long rectangular shape, and its length direction is parallel to the bottom edge of the main body area 104. The end of the second transition zone 136 away from the main body area 104 is connected to the second edge area 108. The height of the second transition zone 136 is less than or equal to the length of the second edge area 108 and extends in a direction away from the second edge area 108, and the free end of the second transition zone 136 is in an arc shape. The length of the second transition zone 136 is greater than the width of the first edge area 108. The height of the second transition zone 136 corresponds to the height from the end connected to the second edge area 108 to the free end which is arc shaped. The length of the second transition zone 136 corresponds to the length from the end connected to the main body area 104 to the end connected to the second edge area 108.

During use, when the first edge area 106 and the second edge area 108 are bent and deformed relative to the main body area 104, the first transition zone 134 and the second transition zone 136 are directly connected to the first edge area 106 and the second edge area 108 can move accordingly and gradually away from the main body area 104. The arc design of the free ends of the first transition zone 134 and the second transition zone 136 can avoid stress concentration to a certain extent, and prevent the first transition zone 134 and the second transition zone 136 from wrinkling or damage during the bending process. In addition, the gap between the first transition zone 134, the second transition zone 136, and the main body area 104 reserves sufficient space for the bending deformation of the first edge area 106 and the second edge area 108, further improving the fitting performance of the phototherapy pad 102.

The main body area 104 is provided with a first connecting portion 138 and a second connecting portion 140 on both sides of the upper edge in width direction. The first connecting portion 138 is located on the side of the main body area 104 away from the free end of the first edge area 106, and the first connecting portion 138 is spaced apart from the first edge area 106. The second connecting portion 140 is located on the side of the main body area 104 away from the free end of the second edge area 108, and the second connecting portion 140 is spaced apart from the second edge area 108. It should be noted that there is a gap between the first connecting portion 138 and the first edge area 106, and the two are not directly connected. The structure and arrangement of the second connecting portion 140 are similar to those of the first connecting portion 138, and it is located on the side of the main body area 104 away from the free end of the second edge area 108, and there is also a gap between the second connecting portion 140 and the second edge area 108. The first connecting portion 138 and the second connecting portion 140 can be conveniently connected with the fixing belt and can be deformed when worn by the user so as to fit more closely to the user's body surface.

In an embodiment, the phototherapy pad 102 includes a light-transmitting layer 120, a substrate layer 128, and a light source assembly 124 for phototherapy. The substrate layer 128 is connected to the light-transmitting layer 120, and the light source assembly 124 is installed between the light-transmitting layer 120 and the substrate layer 128. Optionally, the light-transmitting layer 120 and the substrate layer 128 are flexible layers. The substrate layer 128 can be made of white flexible silicone material, the light-transmitting layer 120 can be made of transparent or translucent flexible silicone material, and the light source assembly 124 is installed between the light-transmitting layer 120 and the substrate layer 128.

In an embodiment, the light source assembly 124 is a flexible LED light board or LED light strip. By using a flexible LED light board or LED light strip, the bendability of the phototherapy pad 102 is ensured. The side of the flexible LED light board facing the light-transmitting layer 120 is arranged with an array of light-emitting diodes or LED lamp beads as light-emitting unit 126. Among them, a plurality of light-emitting unit 126 are arranged on each of the main body area 104, the first edge area 106, and the second edge area 108.

In an embodiment, a soft convex ring 132 is also designed on the side of the light-transmitting layer 120 close to the substrate layer 128, and the light emitting unit 126 of the light source assembly 124 is arranged corresponding to the middle position of the soft convex ring 132. This structural design can not only play a flexible protective role for the light emitting unit 126 to avoid local squeezing damage but also improve the uniformity of illumination by uniformly scattering the light emitted by the light emitting unit 126.

A positioning hole is opened on the light source assembly 124, and a positioning protrusion 130 that fits the positioning hole is provided on the substrate layer 128. The role of the positioning protrusion 130 is to realize the positioning of the light source assembly 124 and the substrate layer 116, so as to avoid the light source assembly 124 from being offset during use, which affects the accuracy of illumination.

A control component 118 is installed at one end of the first edge area 106 connected to the main body area 104, and the control component 118 is electrically connected to the light source assembly 124.

In another embodiment, a control component 118 is installed at one end of the second edge area 108 connected to the main body area 104, and the control component 118 is electrically connected to the light source assembly 124. In other embodiments, the control component 118 can also be installed at the position where the first edge area 106 and the second edge area 108 are connected to the main body area 104, so as to control the phototherapy pad.

In one embodiment of the present application, a control component 118 is installed at one end of the first edge area 106 connected to the main body area 104, and the control component 118 includes a switch button, a charging interface, and an LED indicator light. The control component 118 is electrically connected to the light source assembly 124 inside the phototherapy pad 102 through a wire. The control component 118 integrates components such as a PCB circuit board, a battery, and a control chip, and is used to control the working state of the light emitting unit 126 on the flexible LED light board. The user can adjust the lighting mode and intensity by operating the control component 118.

The phototherapy pad 102 also includes a fixing belt 110 for fixing the phototherapy pad 102, and the two ends of the fixing belt 110 are respectively connected to the two sides of the main body area 104. Optionally, the fixing belt 110 is detachably connected to the first belt holder 112, and the second connecting portion 140 is detachably connected to the second belt holder 114, and the fixing belt 110 is connected to the first belt holder 112, and the second belt holder 114. The first connecting portion 138 is provided with a first buttonhole, and the first belt holder 112 is provided with a first fastener, and the first fastener is buckled in the first buttonhole to achieve the connection between the first belt holder 112 and the first connecting portion 138. The second connecting portion 140 is provided with a second buttonhole, and the second belt holder 114 is provided with a second fastener, which is buckled in the second buttonhole to achieve the connection between the second belt holder 114 and the second connecting portion 140.

Optionally, a strip groove perpendicular to the length direction of the first belt holder 112 and the second belt holder 114 is provided for connecting the fixing belt 110. The fixing belt 110 can be made of elastic webbing or silicone strip, and its width matches the width of the strip groove. The two ends of the fixing belt 110 pass through the strip grooves of the first belt holder 112 and the second belt holder 114, respectively, and the ends of the fixing belt 110 can be fastened by a buckle. When in use, one end of the fixing belt 110 is passed through the strip groove on the first belt holder 112, and then the fixing belt is passed around the human body, and the other end is passed through the strip groove on the second belt holder 114 in the same way, and finally the two ends of the fixing belt 110 are fastened, so that the phototherapy pad 102 can be firmly fixed to the part of the human body that needs to be treated. Patients can adjust the tightness of the end of the fixing belt 110 to adapt to the physiological curvature of different parts and obtain a more comfortable wearing experience.

The material of the first belt holder 112 and the second belt holder 114 can be selected from soft and breathable materials such as fabric or silicone, and its thickness is usually less than the thickness of the main body area 104. During the treatment process, the first belt holder 112 and the second belt holder 114 can be deformed together with the main body area 104, while the first edge area 106 and the second edge area 108 are free to bend on the side away from the first belt holder 112 and the second belt holder 114 to fit the local curved surface of the human body.

In an embodiment of the present invention, a plastic reinforcement layer 122 is added between the light-transmitting layer 120 and the light source assembly 124. The plastic reinforcement layer 122 is injection molded with a transparent PC material. The strength and hardness of the plastic reinforcement layer 122 are higher than those of the light-transmitting layer 120 and the light source assembly 124, which plays a role in improving the overall rigidity and support strength of the phototherapy pad 102. The shape of the plastic reinforcement layer 122 is substantially the same as that of the phototherapy pad 102. The plastic reinforcement layer 122 is provided with a circular through hole corresponding to the position of the light emitting unit 126 on the light source assembly 124. The diameter of the circular through hole is slightly larger than the diameter of the light emitting unit 126, which can avoid blocking the light of the light emitting unit 126 and limit the movement of the light emitting unit 126 in the hole.

The plastic reinforcement layer 122 can be bonded to the light-transmitting layer 120 and the light source assembly 124 by glue. During the bonding process, vacuum assistance can be used to discharge the air in the bonding interface and the through hole to improve the bonding strength and optical transmittance. When in use, the plastic reinforcement layer 122 is located between the light-transmitting layer 120 and the light source assembly 124. When it is bent and deformed, the deformation of the remaining parts is relatively small except for the edge, so it will not squeeze the soft convex ring 132 above the light-transmitting layer 120. The plastic reinforcement layer 122 added in this embodiment not only improves the overall strength of the phototherapy pad 102, but also uses the precisely opened through hole to realize the position limitation of the light emitting unit 126. It can be seen that this embodiment, on the basis of giving full play to the flexible fitting characteristics of the phototherapy pad 102, further strengthens its overall strength, making it more reliable and durable in clinical applications.

Various modifications to these embodiments are apparent to those skilled in the art, from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended claims.

1. A phototherapy pad, comprising:
a main body area configured to cover a treatment area, the main body area comprises an upper end, a lower end a first side, and a second side connecting the upper end and the lower end;
a first edge area having a first end connected to the upper end of the main body area and extending laterally along the first side of the main body area towards the lower end; and
a second edge area having a first end connected to the upper end of the main body area and extending laterally along the second side of the main body area towards the lower end.

2. The phototherapy pad of claim 1, wherein the main body area further comprises a first transition zone located on the first side of the main body area, the first edge area is connected to the first transition zone; and height of the first transition zone is less than or equal to the length of the first edge area.

3. The phototherapy pad of claim 2, wherein the length of the first transition zone is greater than the width of the first edge area.

4. The phototherapy pad of claim 1, wherein the main body area further comprises a second transition zone located on the second side of the main body area, the second edge area is connected to the second transition zone; and height of the second transition zone is less than or equal to the length of the second edge area.

5. The phototherapy pad of claim 4, wherein the length of the second transition zone is greater than the width of the second edge area.

6. The phototherapy pad of claim 1, wherein the main body further comprises a first connecting portion at the first side and a second connecting portion at the second side on the upper end of the main body area.

7. The phototherapy pad of claim 6, further comprising a fixing belt to secure the phototherapy pad, the fixing belt is connected to the first connecting portion and the second connecting portion.

8. The phototherapy pad of claim 1, wherein the phototherapy pad comprises a light-transmitting layer, a substrate layer, and a light source assembly for providing phototherapy; the substrate layer is connected to the light-transmitting layer, and the light source assembly is installed between the light-transmitting layer and the substrate layer.

9. The phototherapy pad of claim 8, wherein the light-transmitting layer and the substrate layer are both flexible; and the light source assembly comprises a flexible LED light board or LED light strip.

10. The phototherapy pad of claim 8, wherein the light-transmitting layer comprises a convex ring at side facing the substrate layer, and a light-emitting unit is positioned at the center of the convex ring.

11. The phototherapy pad of claim 8, wherein a positioning hole is provided in the light source assembly, and a positioning protrusion is provided on the substrate layer to fit into the positioning hole.

12. The phototherapy pad of claim 8, wherein a control assembly is provided in the first edge area or the second edge area, the control assembly is electrically connected to the light source assembly.
